(19) United States
(12) Patent Application Publication
(10) Pub. No.: US 2025/0146956 A1
(43) Pub. Date: May 8, 2025

(54) RADIATION-MEASUREMENT-INSTRUMENT SUPPORT DEVICE, RADIATION MEASUREMENT APPARATUS, AND RADIATION MEASUREMENT METHOD

(71) Applicants: KABUSHIKI KAISHA TOSHIBA, Tokyo (JP); TOSHIBA ENERGY SYSTEMS & SOLUTIONS CORPORATION, Kawasaki-shi, Kanagawa (JP)

(72) Inventor: Ryoutaro MASUDA, Yokohama Kanagawa (JP)

(73) Assignees: KABUSHIKI KAISHA TOSHIBA, Tokyo (JP); TOSHIBA ENERGY SYSTEMS & SOLUTIONS CORPORATION, Kawasaki-shi (JP)

(21) Appl. No.: 19/005,279

(22) Filed: Dec. 30, 2024

Related U.S. Application Data
(63) Continuation of application No. PCT/JP2023/018087, filed on May 15, 2023.

(30) Foreign Application Priority Data
Aug. 30, 2022 (JP) 2022-136492

(51) Int. Cl. G01N 23/02

(52) U.S. Cl. CPC G01N 23/02; G01N 2223/104; G01N 2223/3035; G01N 2223/309; G01N 2223/502

(57) ABSTRACT

According to one embodiment, a radiation-measurement-instrument support device comprising: a cylindrical casing configured to house at least one phantom and a radiation measurement instrument and formed in a cylindrical shape; a base configured to rotatably support the cylindrical casing in a circumferential direction in a state where a cylindrical axis of the cylindrical casing is directed in a horizontal direction and fix the cylindrical casing at an arbitrary rotation angle in the circumferential direction; and an angle display configured to display the rotation angle.

### CROSS-REFERENCE TO RELATED APPLICATIONS

This application is a Continuation Application of No. PCT/JP2023/018087, filed on May 15, 2023, and the PCT application is based upon and claims the benefit of priority from Japanese Patent Application No. 2022-136492, filed on Aug. 30, 2022, the entire contents of which are incorporated herein by reference.

### TECHNICAL FIELD

Embodiments of the present invention relate to radiation measurement techniques.

### BACKGROUND

In a particle beam therapy system provided with a rotating gantry, its irradiation port rotates around a patient, and a particle beam (radiation or radioactive rays) is radiated from an arbitrary direction depending on a treatment site of the patient. In such a particle beam therapy system, radiation measurement depending on the position of the irradiation port is required before starting treatment in order to check quality of the particle beam, such as energy, beam size, accuracy of an irradiation position, and dose distribution.

In a conventionally known technique, a phantom and a radiation measurement instrument are fixed to the irradiation port by using an attachment, and the particle beam (radiation) emitted from the irradiation port is measured. However, it takes time and effort to fix the phantom and the radiation measurement instrument to the irradiation port.

### SUMMARY

### Problem to be Solved by Invention

The present invention aims to provide a radiation measurement technique by which work of installing a radiation measurement instrument depending on a position of an irradiation port can be facilitated in a particle beam therapy system provided with a positionally changeable irradiation port.

### BRIEF DESCRIPTION OF DRAWINGS

FIG. 1 is a side view illustrating a rotating gantry of a particle beam therapy system.

FIG. 2 is a front view illustrating the rotating gantry corresponding to the cross-section taken along the line II-II in FIG. 1.

FIG. 3 is a front view illustrating the rotating gantry when the position of the irradiation port changes due to rotation of the rotating gantry.

FIG. 4 is a front view illustrating a radiation-measurement-instrument support device according to the first embodiment.

FIG. 5 is a side view illustrating the radiation-measurement-instrument support device according to the first embodiment.

FIG. 6 is a bottom view illustrating the radiation-measurement-instrument support device according to the first embodiment.

FIG. 7 is a perspective view illustrating a frame unit.

FIG. 8 is a cross-sectional view illustrating the radiation-measurement-instrument support device when its rotation angle is 0 degrees.

FIG. 9 is a cross-sectional view illustrating the radiation-measurement-instrument support device when its rotation angle is 90 degrees.

FIG. 10 is a cross-sectional view illustrating the radiation-measurement-instrument support device when its rotation angle is 135 degrees.

FIG. 11 is a cross-sectional view illustrating the radiation-measurement-instrument support device when its rotation angle is 180 degrees.

FIG. 12 is a cross-sectional view illustrating the radiation-measurement-instrument support device according to the second embodiment.

FIG. 13 is a cross-sectional view illustrating the radiation-measurement-instrument support device according to the third embodiment.

FIG. 14 is a cross-sectional view illustrating the radiation-measurement-instrument support device according to the fourth embodiment.

FIG. 15 is a cross-sectional view illustrating the radiation-measurement-instrument support device according to the fifth embodiment.

FIG. 16 is a block diagram illustrating the radiation-measurement-instrument support device according to the fifth embodiment.

### DETAILED DESCRIPTION

In one embodiment of the present invention, a radiation-measurement-instrument support device comprising: a cylindrical casing configured to house at least one phantom and a radiation measurement instrument and formed in a cylindrical shape; a base configured to rotatably support the cylindrical casing in a circumferential direction in a state where a cylindrical axis of the cylindrical casing is directed in a horizontal direction and fix the cylindrical casing at an arbitrary rotation angle in the circumferential direction; and an angle display configured to display the rotation angle.

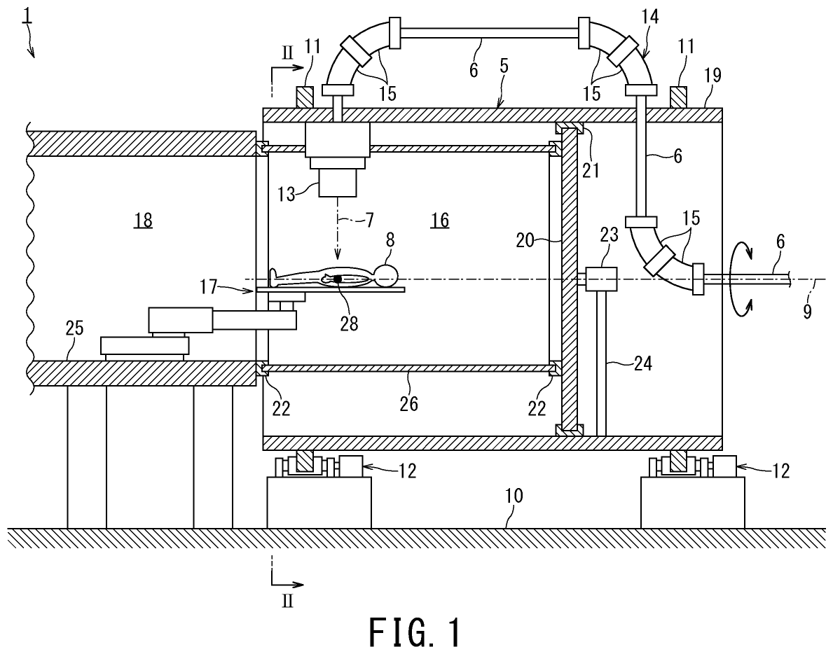

According to embodiments of the present invention, it is possible to provide a radiation measurement technique by which work of installing a radiation measurement instrument depending on a position of an irradiation port can be facilitated in a particle beam therapy system provided with a positionally changeable irradiation port.

[First Embodiment] Hereinbelow, respective embodiments of a radiation-measurement-instrument support device, a radiation measurement apparatus, and a radiation measurement method will be described in detail by referring to the accompanying drawings. The first embodiment will be described by using FIG. 1 to FIG. 11.

The reference sign 1 in FIG. 1 denotes a particle beam therapy system. This particle beam therapy system 1 includes a rotating gantry 5. The particle beam therapy system 1 performs treatment by irradiating a focal tissue (cancer) of a patient 8 to be treated as an object with a particle beam 7 (therapeutic radiation), such as carbon ions, transported through vacuum ducts 6.

A radiation therapy technique with the use of the particle beam therapy system 1 is also referred to as a heavy ion beam cancer treatment technique. This technique is said to be able to damage a cancerous lesion (i.e., focus of disease) and minimize the damage to normal cells by pinpointing the cancerous lesion with carbon ions. Note that the particle beam 7 is defined as radioactive rays heavier than electron, and include proton beam and heavy ion beam, for example. Of these particle beams, heavy ion beam is defined as radioactive rays heavier than helium atoms.

As compared with the conventional cancer treatment using X-rays, gamma rays, or proton beam, the cancer treatment using heavy ion beam has characteristics that: (i) the ability to kill the cancerous lesion is higher; and (ii) the radiation dose is weak on the surface of the body of the patient 8 so as to peak at the cancerous lesion. Thus, the number of irradiations and side effects can be reduced, and the treatment period can be shortened.

The particle beam therapy system 1 includes a beam generator (not shown), a circular accelerator (not shown), and a beam transport line (not shown).

The beam generator has an ion source of carbon ions, which are charged particles, and uses these carbon ions to generate a particle beam 7. The circular accelerator has a ring shape in a plan view, and accelerates the particle beam 7 generated by the beam generator. The beam transport line 4 transports the particle beam 7 accelerated by the circular accelerator 3 to the rotating gantry 5. The patient 8 to be irradiated with the particle beam 7 is placed in the rotating gantry 5.

In this particle beam therapy system 1, first, the particle beam 7 of carbon ions generated by the beam generator is inputted from the beam generator to the circular accelerator. This particle beam 7 is accelerated to approximately 70% of the speed of light while orbiting the circular accelerator approximately one million times. Thereafter, this particle beam 7 is guided to the rotating gantry 5 via the beam transport line.

The beam generator, the circular accelerator, and the beam transport line are provided with the vacuum ducts 6 (beam pipes), inside of which is vacuumized. The particle beam 7 passes the inside of the vacuum ducts 6. The vacuum ducts 6 of the beam generator, the circular accelerator, and the beam transport line are integrated so as to form a transport path that guides the particle beam 7 to the rotating gantry 5. In other words, the vacuum ducts 6 are closed continuous space with a sufficient degree of vacuum to allow the particle beam 7 to pass through.

As shown in the cross-sectional view of FIG. 1, the rotating gantry 5 is an apparatus in a cylindrical shape. This rotating gantry 5 is installed in such a manner that a horizontal axis 9 of its cylindrical body is directed in the horizontal direction. The rotating gantry 5 can rotate around this horizontal axis 9.

The rotating gantry 5 is supported by a structure 10 of a building constituting a treatment facility in which the particle beam therapy system 1 is installed. For example, end rings 11 are fixed to the front portion and the rear portion of a main unit 19 of the rotating gantry 5. Below these end rings 11, rotary drivers 12 are provided. The rotary drivers 12 rotatably support the end rings 11 and include drive motors. These rotary drivers 12 are supported by the structure 10. The driving force of the rotary drivers 12 is applied to the rotating gantry 5 through the end rings 11, and thereby, the rotating gantry 5 is rotated around the horizontal axis 9.

The rotating gantry 5 is provided with the vacuum ducts 6 extending from the beam transport line. The vacuum ducts 6 are first guided from the rear side of the rotating gantry 5 into the inside along the horizontal axis 9. Further, the vacuum ducts 6 once extend outward from the outer circumferential surface of the rotating gantry 5, and then again extend toward the inside of the rotating gantry 5. The tip of the vacuum ducts 6 extends to a position close to the patient 8.

Of the vacuum ducts 6, the portion along the horizontal axis 9 of the rotating gantry 5 is provided with a predetermined rotation mechanism, which is not particularly illustrated. Of the vacuum ducts 6, the portion outside this rotating mechanism is stationary, and the portion inside this rotating mechanism rotates together with the rotation of the rotating gantry 5.

In addition, the rotating gantry 5 includes: an irradiation port 13 configured to irradiate the patient 8 with the particle beam 7; and a transport unit 14 configured to transport the particle beam 7 to the irradiation port 13. In other words, the irradiation port 13 and the transport unit 14 are supported by the rotating gantry 5.

Further, the transport unit 14 includes superconducting electromagnets 15 configured to generate a magnetic field that forms a path for transporting the particle beam 7. These superconducting electromagnets 15 are bending electromagnets configured to change the traveling direction of the particle beam 7 along the vacuum ducts 6 or quadrupole electromagnets configured to control convergence and divergence of the particle beam 7, for example.

The irradiation port 13 is provided at the tip of the vacuum ducts 6 and radiates the particle beam 7 guided by the transport unit 14 toward the patient 8. The irradiation port 13 is fixed to the inner circumferential surface of the rotating gantry 5. Note that the particle beam 7 is radiated from the irradiation port 13 in the direction perpendicular to the horizontal axis 9.

Inside the rotating gantry 5, a treatment space 16 for performing particle beam therapy is provided. The patient 8 is placed on a treatment table 17 provided in this treatment space 16. This treatment table 17 can be moved with the patient 8 placed thereon. Positioning can be performed by moving this treatment table 17 in such a manner that the patient 8 on this treatment table 17 is moved to the irradiation position of the particle beam 7. Thus, the particle beam 7 can be radiated to an appropriate site such as the diseased tissue of the patient 8.

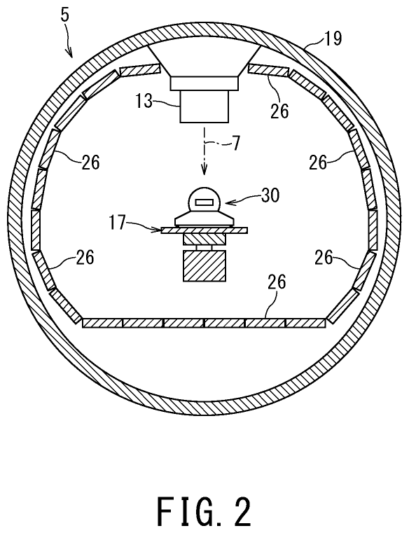

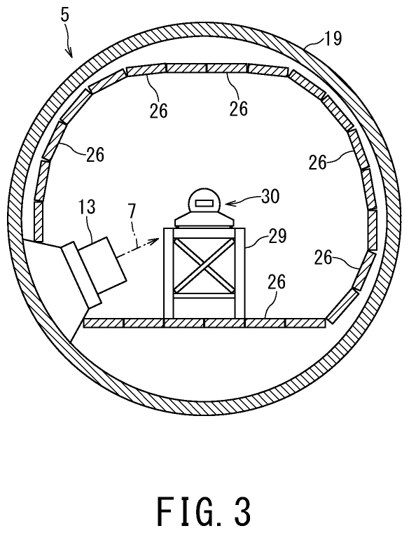

The patient 8 is placed at the position of the horizontal axis 9, and the irradiation port 13 can be rotated around the stationary patient 8 by rotating the rotating gantry 5. For example, the irradiation port 13 can be rotated around the patient 8 (i.e., around the horizontal axis 9) clockwise or counterclockwise in increments of 180° when viewed from the front (FIG. 2 and FIG. 3). The particle beam 7 can be radiated from any direction around the patient 8. In other words, the rotating gantry 5 is an apparatus that can change the irradiation direction of the particle beam 7 guided by the beam transport line 4 with respect to the patient 8. Thus, the particle beam 7 can be radiated from the appropriate direction to the lesion site with higher precision while reducing the burden on the patient 8.

The particle beam 7 loses its kinetic energy at the time of passing through the body of the patient 8 so as to decrease its velocity and receive a resistance that is approximately inversely proportional to the square of the velocity, and stops rapidly when it decreases to a certain velocity. The stopping point of the particle beam 7 is referred to as the Bragg peak at which high energy is emitted. The particle beam therapy system 1 matches this Bragg peak with the position of the lesion tissue (i.e., affected part) of the patient 8, and thus, can kill only the lesion tissue while suppressing the damage to normal tissues.

As shown in FIG. 1, in the treatment space 16, a virtual point is set as the position where the particle beam 7 is most concentrated. This virtual point is called an isocenter 28. At the time of treatment, a diseased site of the patient 8 is placed at this isocenter 28. The isocenter 28 is located at the horizontal axis 9 of the rotating gantry 5, for example. The position of the isocenter 28 does not change regardless of rotation of the rotating gantry 5.

The treatment space 16 provided inside the rotating gantry 5 is formed integrally with a treatment room 18 located on the front side of the rotating gantry 5. The floor, ceiling, and walls of the treatment room 18 are constituted by a building-side structure 25 supported by the structure 10 of the building. Note that the treatment table 17 is supported by this building-side structure 25. In other words, it is configured such that the position of the treatment table 17 does not change even if the rotating gantry 5 and the irradiation port 13 are rotated.

An inner wall portion 20 as a decorative plate is provided inside the rotating gantry 5. This inner wall portion 20 has a disk shape, and its circumferential edge is supported by a support rail 21 provided over the entirety of the inner circumferential surface of the rotating gantry 5. This inner wall portion 20 is supported by the support rail 21 so as to be rotatable in the circumferential direction.

Of the inner wall portion 20, the central portion on the side opposite to the treatment space 16 is connected to a counter-rotating synchronous motor 23. This counter-rotating synchronous motor 23 is fixed to the inner circumferential surface of the rotating gantry 5 via a support rod 24. When the rotating gantry 5 rotates, driving the counter-rotating synchronous motor 23 causes the inner wall portion 20 to rotate in the direction opposite to the rotation direction of the rotating gantry 5 due to driving force of the counter-rotating synchronous motor 23.

For example, when the rotating gantry 5 rotates clockwise in the front view, the inner wall portion 20 is rotated counterclockwise. At this time, the rotational speed of the rotating gantry 5 and the rotational speed of the inner wall portion 20 are controlled so as to be the same. In other words, the inner wall portion 20 appears to remain stationary even when the rotating gantry 5 is rotated.

Of the inner wall portion 20, the side facing the treatment space 16 is provided with a track rail 22, i.e., this track rail 22 is fixed to the side facing the treatment space 16. Of the building-side structure 25, the side facing the treatment space 16 is provided with another track rail 22, i.e., this track rail 22 is fixed to the side facing the treatment space 16. These track rails 22 are installed so as to be positionally unchanged regardless of rotation of the rotating gantry 5 and rotation of the irradiation port 13. A plurality of movable floors 26 are held between these track rails 22. Each movable floor 26 is a rectangular plate-shaped member. The respective movable floors 26 are arranged in line along the inner circumferential surface of the rotating gantry 5, and both ends of each movable floor 26 are held by the track rails 22.

As shown in FIG. 2 and FIG. 3, the floor, walls, and ceiling of the treatment space 16 are formed by the plurality of movable floors 26. These movable floors 26 move together with the irradiation port 13 when the rotating gantry 5 and the irradiation port 13 are rotated. Regardless of the position to which the irradiation port 13 is moved, the movable floors 26 maintain the floor, walls, and ceiling of the treatment space 16.

Of each movable floor 26, the portion corresponding to the floor is provided so as to be at the same height and on the same plane as the floor of the treatment room 18. At the time of preparing for treatment, the patient 8 and a technologist can walk on the floor surface formed by these movable floors 26. Since the movable floors 26 always form the floor, walls, and ceiling of the treatment space 16, the inner circumferential surface of the rotating gantry 5 is concealed so as to be invisible to the patient 8.

Next, the radiation-measurement-instrument support device 30 according to the first embodiment will be described. In the particle beam therapy system 1, before starting treatment, radiation measurement is performed depending on the position of the irradiation port 13 in order to check the quality of the particle beam 7, such as energy, beam size, irradiation position accuracy, and dose distribution. For example, radiation measurement is performed at each rotational position by rotating the irradiation port 13 clockwise or counterclockwise by a predetermined angle when viewed from the front. In order to perform such radiation measurement, the radiation-measurement-instrument support device 30 is used.

This radiation-measurement-instrument support device 30 is installed on the upper surface of the treatment table 17 or the pedestal 29. The size and weight of the radiation-measurement-instrument support device 30 are light or compact enough so that the radiation-measurement-instrument support device 30 can be carried by about two users. For example, the total weight of the radiation-measurement-instrument support device 30 is designed to be within 30 kg.

The radiation measurement apparatus according to the first embodiment is composed of at least the radiation-measurement-instrument support device 30, water equivalent phantoms 40, and a radiation measurement instrument 41.

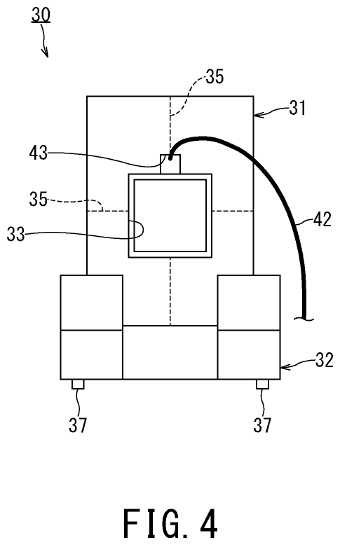

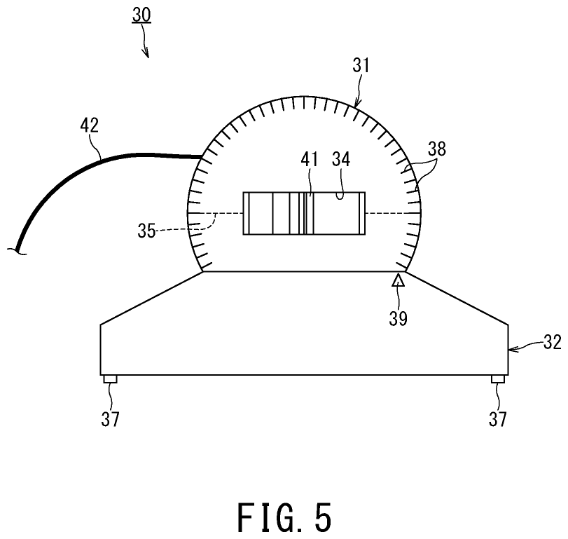

As shown in FIG. 4 and FIG. 5, the radiation-measurement-instrument support device 30 according to the first embodiment includes a cylindrical casing 31 and a base 32.

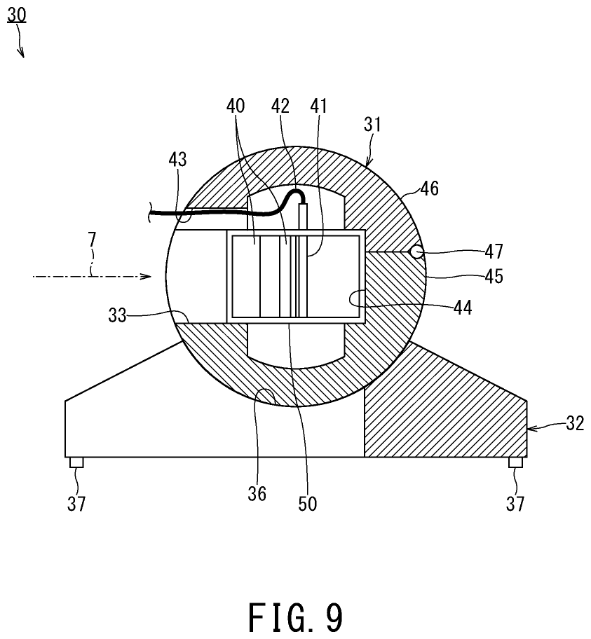

The cylindrical casing 31 has a cylindrical shape. An entrance window 33 through which radiation (i.e., the particle beam 7) enters is opened in a part of the cylindrical circumferential surface (i.e., front surface) of this cylindrical casing 31. In addition, a slit 34 for checking the position of the radiation measurement instrument 41 (FIG. 9) from the outside is opened in a part of the cylindrical bottom surface (i.e., side surface) of the cylindrical casing 31. Further, a plurality of reference lines 35 are drawn on the surface of the cylindrical casing 31. This cylindrical casing 31 is supported by the base 32 under the state where its cylindrical axis is oriented in the horizontal direction.

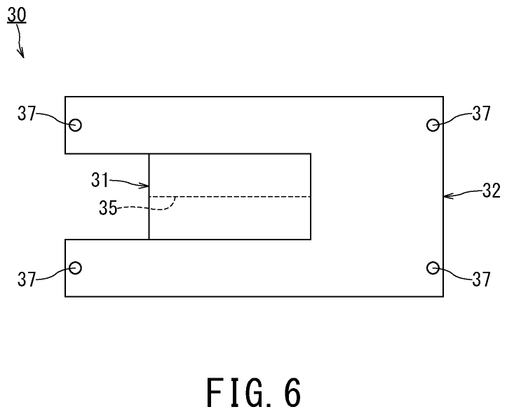

The base 32 supports the cylindrical casing 31 such that the cylindrical casing 31 can rotate in the circumferential direction, and fixes the cylindrical casing 31 at an arbitrary rotation angle in the circumferential direction. A round portion 36 (FIG. 9) into which the cylindrical casing 31 fits from above is formed in the upper portion of the base 32, and this round portion 36 is curved along a part of the circumference of the cylindrical casing 31. In this configuration, the cylindrical casing 31 can be supported in a rotatable state. Further, the base 32 has a U-shape in a bottom view with a portion cut out (FIG. 6). Note that the user can manually rotate the cylindrical casing 31 and fix it at an arbitrary rotation angle.

The base 32 can be installed on the upper surface of the treatment table 17 or the pedestal 29 (FIG. 2 and FIG. 3). Adjusters 37 are provided at the respective four corners of the bottom surface of the base 32. It is satisfactory that the adjuster 37 is provided at each of three or more locations on the bottom surface of the base 32. These adjusters 37 enable fine adjustment of the height. In this manner, the base 32 can be stably placed on the upper surface of the treatment table 17 or the pedestal 29.

The pedestal 29 is installed on the floor portions of the movable floors 26 under the state where the treatment table 17 is evacuated from the treatment space 16. This pedestal 29 allows the radiation-measurement-instrument support device 30 to be installed at an arbitrary height position.

The radiation-measurement-instrument support device 30 further includes an angle display configured to display the rotation angle of the cylindrical casing 31. The angle display of the first embodiment includes: a scale 38 provided on the circumferential edge of the cylindrical bottom surface (i.e., side surface) of the cylindrical casing 31; and a reader 39 that is provided on the base 32 and serves as a reference at the time of reading the scale 38. In this configuration, the user can check the rotation angle of the cylindrical casing 31 by using the scale 38. For example, the scale 38 is provided in increments of 0.5°. Its accuracy is within +0.5°.

The reader 39 is, for example, an arrow or a triangular mark. The number on the scale 38 located at the position of the reader 39 indicates the rotation angle of the cylindrical casing 31.

The water equivalent phantoms 40 and the radiation measurement instrument 41 are housed inside the cylindrical casing 31 (FIG. 9).

The radiation measurement instrument 41 according to the first embodiment is composed of a two-dimensional detector in which a plurality of detection elements are two-dimensionally arranged to form a plate shape. In this configuration, radiation can be measured in a two-dimensionally expanded range.

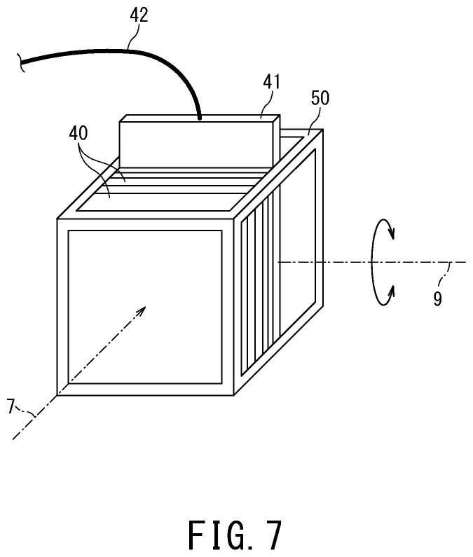

In the first embodiment, solid plate-shaped water equivalent phantoms 40 (FIG. 7) are used as the phantoms. Each water equivalent phantom 40 is made of acrylic resin, for example. A plurality of water equivalent phantoms 40 are stacked one on top of the other, and these water equivalent phantoms 40 and the radiation measurement instrument 41 are arranged and housed inside the cylindrical casing 31.

The respective water equivalent phantoms 40 do not have to be the same in thickness. For example, water equivalent phantoms 40 having a plurality of different thicknesses may be used. These water equivalent phantoms 40 are combined to form a phantom having a desired thickness as a whole. For example, the total thickness of the plurality of water equivalent phantoms 40 can be adjusted within a range of 2 mm to 300 mm. Note that the water equivalent phantoms 40 are stacked in such a manner that no gaps are created between them.

Inside the cylindrical casing 31, a holding portion configured to hold the plurality of water equivalent phantoms 40 and the radiation measurement instrument 41 is formed. In this manner, adjustment of the number of water equivalent phantoms 40 enables adjustment of the total thickness, and thereby, the transmittance (i.e., transmission factor) of radiation (i.e., the particle beam 7) can be arbitrarily adjusted. In other words, radiation can be measured under predetermined arbitrary measurement conditions.

Inside the cylindrical casing 31, a groove portion 43 extending from the holding portion 44 toward the entrance window 33 is also formed. Along this groove 43, a cable 42 extending from the radiation measurement instrument 41 is led out of the cylindrical casing 31.

As shown in FIG. 7, the radiation-measurement-instrument support device 30 according to the first embodiment includes a frame unit 50 for attaching and detaching the water equivalent phantoms 40 and the radiation measurement instrument 41 to/from the cylindrical casing 31 in an integrated state. In this manner, the work of attaching/detaching the water equivalent phantoms 40 and the radiation measurement instrument 41 to/from the cylindrical casing 31 can be readily performed.

The frame unit 50 is formed by combining a plurality of frames such that the frame unit 50 has a rectangular parallelepiped shape as a whole. For example, the water equivalent phantoms 40 and the radiation measurement instrument 41 are inserted into the frame unit 50 from above into the inside.

The radiation-measurement-instrument support device 30 (FIG. 2 and FIG. 3) according to the first embodiment is used for locating the radiation measurement instrument 41 at a position that coincides with the horizontal axis 9 (FIG. 1) of the rotating gantry 5. For example, the cylindrical axis of the cylindrical casing 31 is located at a position that coincides with the horizontal axis 9 of the rotating gantry 5. Rotating the cylindrical casing 31 can direct the radiation measurement instrument 41 in an arbitrary direction while keeping the radiation measurement instrument 41 aligned with the horizontal axis 9. The radiation measurement instrument 41 does not necessarily need to be located at a position that coincides with the horizontal axis 9 of the rotating gantry 5, and may be disposed to be slightly shifted from the incident direction of the particle beam 7.

As shown in FIG. 9, the frame unit 50 is fitted into the holding portion 44 of the cylindrical casing 31. For example, the cylindrical casing 31 is configured by connecting a main body 45 and a lid 46 with the use of a hinge 47. When the lid 46 is swung to open the main body 45, the holding portion 44 is exposed. When the user fits the frame unit 50 into the holding portion 44 and closes the lid 46 again, the water equivalent phantoms 40 and the radiation measurement instrument 41 are housed inside the cylindrical casing 31.

The water equivalent phantoms 40 and the radiation measurement instrument 41 are moved toward the side of the entrance window 33 (i.e., the side of the incident direction of radiation) inside the frame unit 50. For example, when the side facing the entrance window 33 is defined as the front side of the radiation measurement instrument 41, a space is provided on the rear side of the radiation measurement instrument 41. Note that the gap between the rear surface of the rearmost water equivalent phantom 40 and the front surface of the radiation measurement instrument 41 is 1 mm or less.

As shown in FIG. 5, the user can check the position of the interior radiation measurement instrument 41 through the slit 34 opened in the cylindrical bottom surface (i.e., side surface) of the cylindrical casing 31. In this manner, the position of the radiation measurement instrument 41 can be aligned with the isocenter 28 (FIG. 1), which is a virtual point to be irradiated with radiation.

For example, at the time of performing radiation measurement, the user looks through the slit 34 and checks the position of the radiation measurement instrument 41. The user then adjusts the position of the treatment table 17 or the pedestal 29 and adjusts the orientation of the base 32 such that the position of the radiation measurement instrument 41 visible through the slit 34 is aligned with the position of the isocenter 28 (FIG. 1), and thereby adjusts the rotation angle of the cylindrical casing 31.

At the time of the above-described positioning or alignment, a visible-light laser is radiated into the treatment space 16. For example, lasers are radiated from the respective three-dimensional directions (one vertical direction and two horizontal directions). The location where these lasers intersect is shown as the isocenter 28. The user aligns the radiation measurement instrument 41 with the position of the isocenter 28 indicated by the lasers. Note that a level may be used to check the horizontal state of the radiation-measurement-instrument support device 30.

In addition, the user may adjust the rotation angle of the cylindrical casing 31 by adjusting the position of the treatment table 17 or the pedestal 29 and adjusting the orientation of the base 32 such that the plurality of reference lines 35 drawn on the surface of the cylindrical casing 31 match the laser irradiation position.

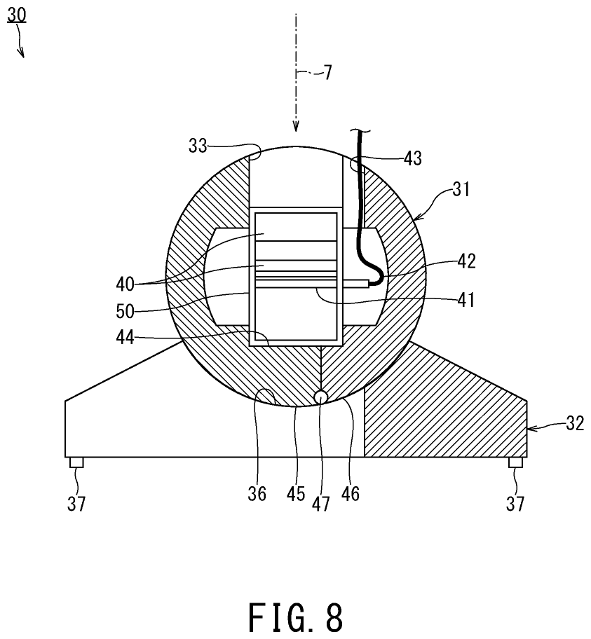

For example, when the irradiation port 13 is located directly above the isocenter 28, i.e., when the irradiation port 13 is at the 0 degree position, the user rotates the cylindrical casing 31 such that the entrance window 33 is at the position directly above it, i.e., the rotation angle is 0 degrees as shown in FIG. 8. Radiation (i.e., the particle beam 7) is then measured at this rotation angle.

When the rotating gantry 5 rotates and the irradiation port 13 is at a position of 90 degrees, the user rotates the cylindrical casing 31 such that the rotation angle becomes 90 degrees as shown in FIG. 9, and then, radiation (i.e., the particle beam 7) is measured at this rotation angle. When the rotating gantry 5 is rotated and the irradiation port 13 is at a position of 270 degrees, the direction of the base 32 is changed horizontally by 180 degrees while the rotation angle of the cylindrical casing 31 is being kept at 90 degrees. Consequently, the entrance window 33 faces the irradiation port 13.

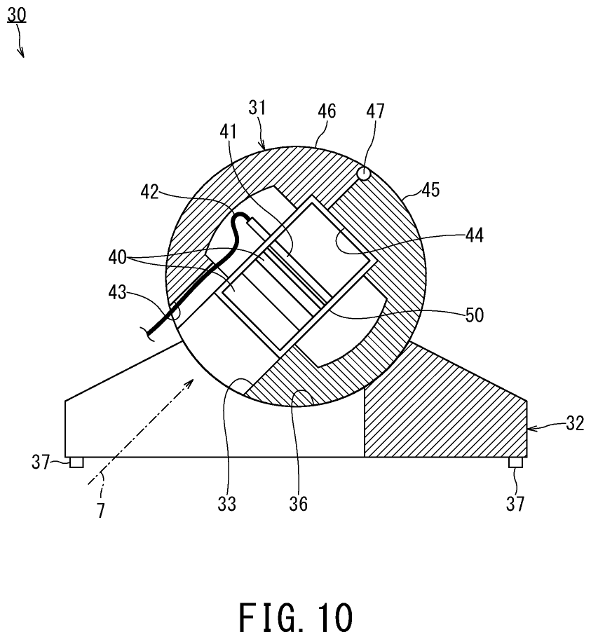

When the rotating gantry 5 rotates and the irradiation port 13 is at a position of 135 degrees, the user rotates the cylindrical casing 31 such that the rotation angle becomes 135 degrees as shown in FIG. 10. Since the base 32 has the U-shape when viewed from the bottom, the entrance window 33 faces the irradiation port 13 even at this rotation angle. Radiation (i.e., the particle beam 7) is measured at this rotation angle.

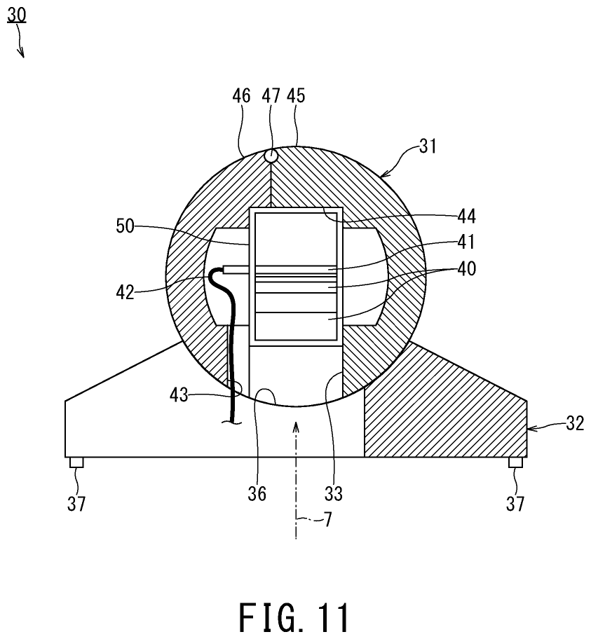

When the rotating gantry 5 rotates and the irradiation port 13 is at a position of 180 degrees, the user rotates the cylindrical casing 31 such that the rotation angle becomes 180 degrees as shown in FIG. 11. Since the base 32 has the U-shape in a bottom view and is opened on its directly lower side, the entrance window 33 faces the irradiation port 13 even at this rotation angle. Radiation (i.e., the particle beam 7) is measured at this rotation angle.

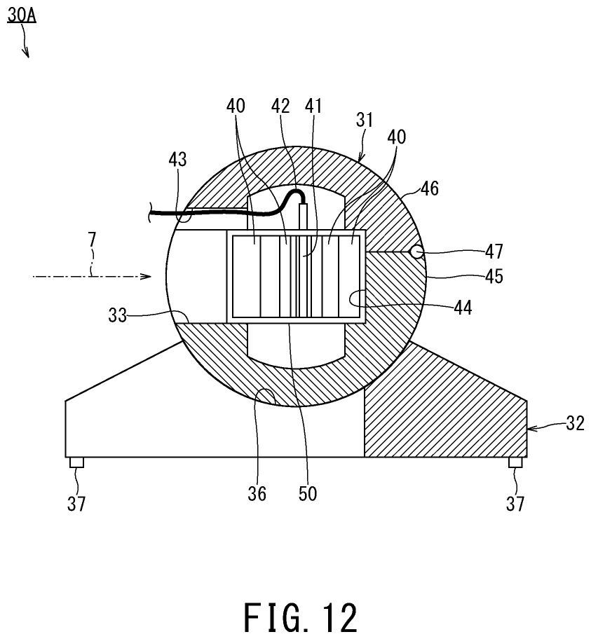

In the radiation measurement method according to the first embodiment, first, the user rotates the rotating gantry 5 and thereby moves the irradiation port 13 to an arbitrary desired position for measurement. Next, the user installs the radiation-measurement-instrument support device 30 inside the rotating gantry 5 of the particle beam therapy system 1. Next, the user adjusts the orientation of the radiation measurement instrument 41 by rotating the cylindrical casing 31 in accordance with rotation of the rotating gantry 5. Afterward, radiation (i.e., the particle beam 7) emitted from the irradiation port 13 of the rotating gantry 5 is measured by the radiation measurement instrument 41. Note that these steps are at least part of the radiation measurement method, and other steps may be included in the radiation measurement method.

[Second Embodiment] Next, the second embodiment will be described by referring to FIG. 12. The same reference signs are assigned to the same components as the first embodiment in each figure, and duplicate description is omitted.

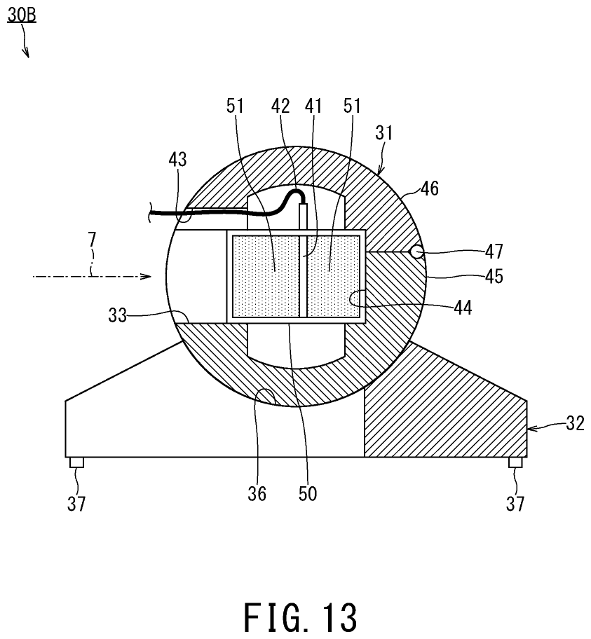

In the radiation-measurement-instrument support device 30A (radiation measurement apparatus) according to the second embodiment, of the cylindrical casing 31, the side facing the entrance window 33 is assumed to be the front side of the radiation measurement instrument 41. Under this assumption, the water equivalent phantoms 40 are provided on both the front side and the rear side of the radiation measurement instrument 41. This configuration can evaluate not only the radiation (i.e., the particle beam 7) to be made incident on the radiation measurement instrument 41 from the front but also the contribution of the radiation to be made incident on the radiation measurement instrument 41 from the rear (i.e., contribution of backscattering).

[Third Embodiment] Next, the third embodiment will be described by referring to FIG. 13. The same reference signs are assigned to the same components as the first embodiment in each figure, and duplicate description is omitted.

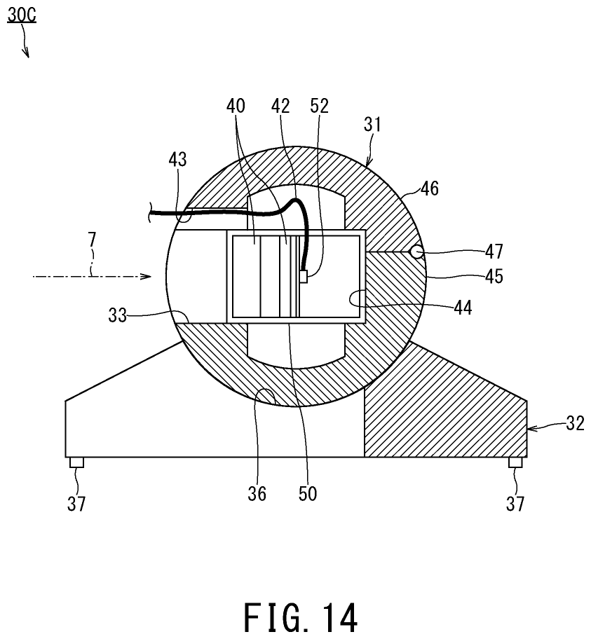

In the radiation-measurement-instrument support device 30B (radiation measurement apparatus) according to the third embodiment, liquid water is used as the phantoms. The radiation-measurement-instrument support device 30B includes containers 51 configured to accommodate water alongside (i.e., side by side) the radiation measurement instrument 41. For example, inside the frame unit 50, respective containers 51 filled with water are provided on the front-surface side and the back-surface side of the radiation measurement instrument 41. Since water is used as the phantoms in this configuration, the phantoms can be provided at low cost. It is not necessarily required to provide a plurality of containers 51, and it may be configured such that one container 51 filled with water is provided only on the front-surface side of the radiation measurement instrument 41.

[Fourth Embodiment] Next, the fourth embodiment will be described by referring to FIG. 14. The same reference signs are assigned to the same components as the first embodiment in each figure, and duplicate description is omitted.

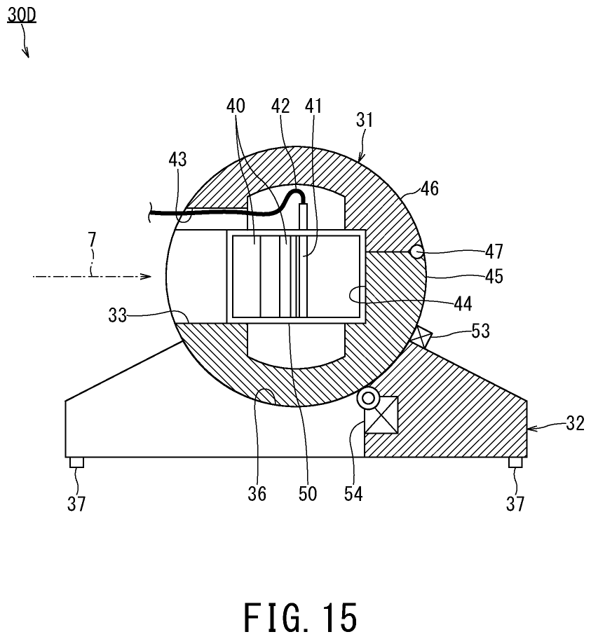

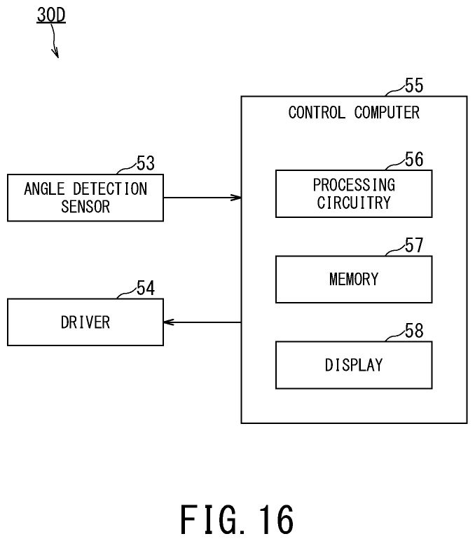

The radiation-measurement-instrument support device 30C (radiation measurement apparatus) according to the fourth embodiment includes an ion chamber 52 instead of the radiation measurement instrument 41 composed of the above-described two-dimensional detector. The ion chamber 52 is an instrument that includes a gas-filled chamber (not shown) and an electrode (not shown) configured to detect ionization of the gas. In this configuration, the instrument for measuring radiation can be provided at low cost. Note that the ion chamber 52 is fixed to the water equivalent phantom(s) 40 at a predetermined position inside the frame unit 50 by using a predetermined jig (not shown).

[Fifth Embodiment] Next, the fifth embodiment will be described by referring to FIG. 15 and FIG. 16. The same reference signs are assigned to the same components as the first embodiment in each figure, and duplicate description is omitted.

As shown in FIG. 15, the radiation-measurement-instrument support device 30D (radiation measurement apparatus) according to the fifth embodiment includes an angle detection sensor 53, a driver 54, and a control computer 55.

The angle detection sensor 53 is provided on the base 32 and detects the rotation angle of the cylindrical casing 31. The driver 54 includes: a roller to be brought in contact with the outer circumferential surface of the cylindrical casing 31; and a motor configured to rotate this roller. This driver 54 is controlled by the control computer 55. In this manner, the orientation of the radiation measurement instrument 41 can be adjusted by automatically rotating the cylindrical casing 31.

The control computer 55 of the present embodiment includes hardware resources such as a Central Processing Unit (CPU), a Graphics Processing Unit (GPU), a Read Only Memory (ROM), a Random Access Memory (RAM), a Hard Disk Drive (HDD) and/or a Solid State Drive (SSD), and is configured as a computer in which information processing by software is achieved with the use of the hardware resources by causing the CPU to execute various programs.

As shown in FIG. 16, the control computer 55 includes at least processing circuitry 56, a memory 57, and a display 58. Note that the control computer 55 may include other components other than these components 56 to 58.

The processing circuitry 56 of the present embodiment is, for example, a circuit provided with a Central Processing Unit (CPU), a Graphics Processing Unit (GPU) and/or a special-purpose or general-purpose processor. The processor implements various functions by executing programs stored in the memory 57. The processing circuitry 56 may be configured of hardware such as a field programmable gate array (FPGA) and an application specific integrated circuit (ASIC). The various functions can also be implemented by such hardware. Additionally, the processing circuitry 56 can implement the various functions by combining hardware processing and software processing based on its processor and programs.

The memory 57 of the control computer 55 stores various information items necessary for executing the radiation measurement method. Moreover, the display 58 outputs predetermined information. This display 58 displays the rotation angle of the cylindrical casing 31.

The display 58 may be separated from the main body of the computer or may be integrated with the main body of the computer. Additionally, or alternatively, the control computer 55 may control images to be displayed on the display of other computers interconnected via the network.

The angle display of the fifth embodiment is composed of the angle detection sensor 53 and the display 58 of the control computer 55. The control computer 55 acquires the rotation angle of the cylindrical casing 31 on the basis of the signal outputted by the angle detection sensor 53. Further, the control computer 55 performs control of displaying this rotation angle on the display 58. In this manner, the user can check the rotation angle of the cylindrical casing 31 on the display 58.

Note that the control computer 55 may acquire the rotation angle of the cylindrical casing 31 on the basis of the control signal of the driver 54 without using the angle detection sensor 53.

In addition, the control computer 55 may control the driver 54 on the basis of the rotation angle of the cylindrical casing 31 acquired by the angle detection sensor 53 so as to: bring the cylindrical casing 31 at a preset target rotation angle; and thereby rotate the cylindrical casing 31.

The control computer 55 includes a control device, a storage device, an output device, an input device, and a communication interface. The control device includes a highly integrated processor such as a Central Processing Unit (CPU), a Graphics Processing Unit (GPU), a Field Programmable Gate Array (FPGA), and a special-purpose chip. The storage device includes a Read Only Memory (ROM), a Random Access Memory (RAM), a Hard Disk Drive (HDD), a Solid State Drive (SSD), or the like. The output device includes a display panel, a head-mounted display, a projector, a printer, or the like. The input device includes a mouse, a keyboard, a touch panel, or the like. The control computer 55 can be achieved by hardware configuration with the use of the normal computer.

Note that the program executed in the control computer 55 is provided by being incorporated in a memory such as the ROM in advance. Additionally, or alternatively, the program may be provided by being stored as a file of installable or executable format in a non-transitory computer-readable storage medium such as a CD-ROM, a CD-R, a memory card, a DVD, and a flexible disk (FD).

In addition, the program executed in the control computer 55 may be stored on a computer connected to a network such as the Internet and be provided by being downloaded via a network. Further, the control computer 55 can also be configured by interconnecting and combining separate modules, which independently exhibit respective functions of the components, via a network or a dedicated line.

As above, although the present invention has been described on the basis of the first to fifth embodiments, the configuration applied in any one of the embodiments may be applied to other embodiments or the configurations in the respective embodiments may be applied in combination.

According to at least one embodiment described above, the base 32 supports the cylindrical casing 31 with the cylindrical axis oriented in the horizontal direction such that the cylindrical casing 31 is rotatable in the circumferential direction, and fixes the cylindrical casing 31 at an arbitrary rotation angle in the circumferential direction. Since such a base 32 is provided, in the particle beam therapy system 1 in which the position of the irradiation port 13 changes, the work of installing the radiation measurement instrument 41 depending on the position of the irradiation port 13 can be readily performed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. The articles “the”, “a” and “an” are not necessarily limited to mean only one, but rather are inclusive and open ended so as to include, optionally, multiple such elements.

1. A radiation-measurement-instrument support device comprising:
a cylindrical casing configured to house at least one phantom and a radiation measurement instrument and formed in a cylindrical shape;
a base configured to
rotatably support the cylindrical casing in a circumferential direction in a state where a cylindrical axis of the cylindrical casing is directed in a horizontal direction and
fix the cylindrical casing at an arbitrary rotation angle in the circumferential direction; and
an angle display configured to display the rotation angle.

2. The radiation-measurement-instrument support device according to claim 1, further comprising a frame unit for attaching and detaching the phantom and the radiation measurement instrument to/from the cylindrical casing in an integrated state.

3. The radiation-measurement-instrument support device according to claim 1, wherein:
an entrance window through which radiation is made incident is opened in a part of a cylindrical circumferential surface of the cylindrical casing;
a holding portion for holding the at least one phantom and the radiation measurement instrument is formed inside the cylindrical casing;
the at least one phantom comprises a plurality of phantoms; and
the phantoms are provided on both a front side and a rear side of the radiation measurement instrument, when a side facing the entrance window is defined as the front side of the radiation measurement instrument.

4. The radiation-measurement-instrument support device according to claim 1, wherein a slit for checking a position of the radiation measurement instrument from outside is opened in a part of a cylindrical bottom surface of the cylindrical casing.

5. The radiation-measurement-instrument support device according to claim 1, wherein the angle display comprises:
a scale provided on a circumferential edge of a cylindrical bottom surface of the cylindrical casing; and
a reader that is provided on the base and serves as a reference at a time of reading the scale.

6. The radiation-measurement-instrument support device according to claim 1, wherein the angle display comprises:
a sensor that detects the rotation angle; and
a computer-display that displays the rotation angle depending on a signal outputted by the sensor.

7. The radiation-measurement-instrument support device according to claim 1, further comprising:
a driver configured to rotate the cylindrical casing; and
a computer configured to control the driver.

8. The radiation-measurement-instrument support device according to claim 1, wherein:
the at least one phantom comprises a plurality of phantoms;
the plurality of phantoms are solid water equivalent phantoms;
a plurality of plate-shaped water equivalent phantoms are stacked one on top of the other; and
a holding portion for holding the water equivalent phantoms and the radiation measurement instrument alongside is formed inside the cylindrical casing.

9. The radiation-measurement-instrument support device according to claim 1, further comprising a container, wherein:
the phantom is water; and
the container is configured to accommodate the water and the radiation measurement instrument alongside.

10. The radiation-measurement-instrument support device according to claim 1, wherein:
the base can be installed on an upper surface of a pedestal;
an adjuster is provided at each of three or more locations on a bottom surface of the base.

11. A radiation measurement apparatus comprising:
the radiation-measurement-instrument support device according to claim 1; and
the radiation measurement instrument including a two-dimensional detector in which a plurality of detection elements are two-dimensionally arranged to form a plate shape.

12. A radiation measurement apparatus comprising:
the radiation-measurement-instrument support device according to claim 1; and
the radiation measurement instrument composed of
a chamber filled with gas and
an ion chamber having an electrode configured to detect ionization of the gas.

13. A radiation measurement method to be executed by using the radiation-measurement-instrument support device according to claim 1, the radiation measurement method comprising steps of:
installing the radiation-measurement-instrument support device inside a rotating gantry of a particle beam therapy system;
adjusting orientation of the radiation measuring instrument by rotating the cylindrical casing in accordance with rotation of the rotating gantry; and
measuring radiation emitted from an irradiation port of the rotating gantry by using the radiation measurement instrument.
